(19) United States
(12) Patent Application Publication
(10) Pub. No.: US 2013/0085577 A1
(43) Pub. Date: Apr. 4, 2013

(54) Prosthesis for Partial Replacement of a Tubular Bone

(71) Applicants: Helmut D. Link, Hamburg (DE); Andreas Dänike, Hamburg (DE); Günther Jendro, Hamburg (DE)

(72) Inventors: Helmut D. Link, Hamburg (DE); Andreas Dänike, Hamburg (DE); Günther Jendro, Hamburg (DE)

(73) Assignee: WALDEMAR LINK GMBH & CO. KG, Hamburg (DE)

(21) Appl. No.: 13/702,228

(22) PCT Filed: Jun. 10, 2011
(86) PCT No.: PCT/EP2011/002875

§ 371 Date: Dec. 5, 2012

(30) Foreign Application Priority Data
Jun. 11, 2010 (EP) 10006098.7

(51) Int. Cl. A61F 2/30

(52) U.S. Cl. CPC A61F 2/30
USPC 623/23.39

(57) ABSTRACT

The invention relates to a prosthesis for at least partial replacement of a tubular bone and of an adjacent joint, comprising an elongate shaft (1) with a first end and a second end, and a joint mechanism (2) arranged at the second end of the shaft (1), wherein a length-adjusting mechanism (3) is provided which actuates the shaft (1) along the axis (10) thereof in the manner of a telescope. The shaft (1) and the joint mechanism (2) are coupled via a plug connection with matching cone connectors (18, 29), wherein the length- adjusting mechanism (3) is of a modular design and, at the proximal and distal ends thereof, is provided with the matching cone plug connectors (18, 19), and it is further provided with an anti-rotation means (35, 37) that acts with a form fit.

The invention relates to a prosthesis for replacing at least part of a tubular bone and an adjoining joint. It comprises an elongate shaft having a first and a second end, as well as a joint mechanism arranged at the second end of the shaft. A length-adjusting mechanism is provided which displaces the shaft along its axis in the manner of a telescope. The invention moreover extends to a prosthesis module system with exchangeable shafts.

Various types of endoprostheses have long been known as a replacement for diseased or defective bones and joints. Prostheses having a shaft extending over the length of the bone to be replaced are used to replace tubular bones, particularly on account of tumour diseases. The shaft replaces and/or reinforces the diseased or absent bone portion. It is frequently connected to a joint mechanism replacing an adjoining joint (for example knee or elbow). The dimensions of the prosthesis shaft must therefore be selected in accordance with the respective anatomy and pathology of the patient.

It is known to offer prostheses in different sizes so as to match individual needs. However, not even with fine grading is it possible to optimally suit the multitude of different needs. This applies all the more to patients who are still growing, i.e. children.

In order to also be able to sufficiently assist those patients, prostheses have been equipped with a length-adjusting mechanism. Thus, a knee prosthesis is known which comprises a shaft and a joint mechanism, with a telescope mechanism being provided in the shaft to change the shaft length (U.S. Pat. No. 4,384,373). In this arrangement, length adjustment of the shaft is possible during surgery. No provision is made for subsequent adjustment.

In order to also be able to adjust the shaft length after surgery, a refined prosthesis is known in which a union nut is provided for actuation (U.S. Pat. No. 4,502,160). It comprises an external toothed ring, the external toothing of which can be actuated by means of a socket key to be laterally inserted. The socket key can be passed through an incision, thus permitting the length of the shaft to be adjusted even after surgery.

To avoid rotation of the prosthesis, and more particularly rotation of the shaft relative to the joint, even when the length is being varied, a rotation lock may be provided (U.S. Pat. No. 4,892,446). A locking screw prevents the shaft from rotating relative to the joint and is loosened to allow for length adjustment.

One disadvantage of these known prostheses is that they are highly specific in each case (actuatable after surgery, secured against rotation, etc.) and therefore only have a narrow field of application.

The invention is based on the object of further developing an endoprosthesis of the kind referred to initially so as to permit its use for a wider range of applications.

The solution according to the invention resides in the features of the independent claims. Advantageous further aspects are the subject matter of the dependent claims.

In a prosthesis for replacing at least part of a tubular bone and an adjoining joint, comprising an elongate shaft having a first and a second end and a joint mechanism arranged at the second end of the shaft, wherein a length-adjusting mechanism is provided which actuates the shaft along its axis in the manner of a telescope, it is provided according to the invention that the shaft and the joint mechanism are coupled via complementary connectors, wherein the length-adjusting mechanism is of a modular design and is provided, at its proximal and distal ends, with the complementary connectors and is further provided with an anti-rotation means acting in a positive fit manner. Complementary is understood to mean that a male connector is provided at one of the two ends and a female connector is provided at the other. The connectors preferably are cone connectors.

The gist of the invention is the idea to design the length-adjusting mechanism in a modular manner and to additionally provide it at its proximal and distal ends with exactly the same type of connectors as are also provided at the transition between the shaft and the prosthesis joint. The length-adjusting mechanism is thus, unlike in the prior art, not an integral part of the prosthesis but can rather be inserted as required. It can, so to speak, be exchanged for a conventional standard shaft member without a length-adjusting mechanism.

The invention thus allows practically any joint prosthesis to be provided with a length-adjusting mechanism in a simple and efficient manner. This significantly improves the adaptability of the prosthesis to the anatomical and/or pathological conditions of the individual patient, without this requiring a large number of different parts having different sizes. The joint mechanism may have practically any design and is able assist or limit movement in the joint to various extents ranging from completely free to stiffened. Due to the fact that, according to the invention, the length-adjusting mechanism is distinct from the actual joint prosthesis as a result of the modular design, the invention can easily also be applied to other prostheses as long as these include corresponding cone connectors. Due to the integrated anti-rotation means acting in a positive fit manner, no further requirements have to be met by the respective basic prosthesis so as to be secured against turning.

The anti-rotation means prevents undesired relative rotation of the shaft and its components. It is a further advantage of the structural integration of securing against turning and length adjustment that the actuating members can be located closely next to one another. Thus, adjustment after surgery requires access from only a narrowly delimited area. A minimally invasive stab incision is sufficient to change the length of the prosthesis. With such a gentle surgical technique, the prosthesis is particularly also suitable for use in children.

Preferably the shaft has an outer and an inner rod acted on by the length-adjusting mechanism. It is thus possible, using a suitable tool, to directly act on the length-adjusting mechanism which correspondingly displaces the outer rod relative to the inner rod in the manner of a telescope.

To ensure sufficient protection against inadvertent adjustment despite the simple adjustability, there is preferably provided a dual securing mechanism which, besides securing against rotation, also forms an adjustment lock by means of two adjacent screws. In this regard, it is furthermore preferred for one of the screws to be non-rotatably disposed on the outer rod and for the other to be rotatably disposed on the adjustment nut.

The outer rod advantageously comprises a compression flange having two opposite collar faces, one of which is a thrust bearing for the conical connection and the other forms a stop for length adjustment. This enables a very compact structure which also allows the length-adjusting mechanism according to the invention to be integrated into relatively small prostheses to be used, for example, on the elbow or the hand.

It may be expedient for a second shaft-length-adjusting mechanism to be provided which is preferably equipped with inversely disposed cone connectors. In the case of long shafts, particularly such used to replace the femur, this also allows the length to be adjusted at the other end. Not only does this extend the range of adjustment, it is also frequently more favourable from a physiological point of view.

The invention furthermore extends to a prosthesis system with several connectable, rigid shaft members of different lengths and a connectable length-adjusting mechanism, wherein preferably at least one of the rigid shaft members is the same length as the length-adjusting mechanism in its initial position. A prosthesis system may thus include prostheses having a shaft of fixed length or a shaft of adjustable length, with it being possible by simply exchanging a rigid shaft module for an adjustable-length shaft module to change from one design to another. This can also be done intraoperatively so that the surgeon may, depending on the circumstances of the case, decide during surgery which variant should be preferably used in the respective case.

According to a particularly advantageous variant which may possibly deserve independent protection, it is provided that in a prosthesis for replacing at least part of a tubular bone there is provided a mechanism for actuating the length-adjusting mechanism which comprises a thread on the inner rod and an adjustment nut which is screwed onto the thread and having a circumferential toothing, wherein on the outer rod there is provided a bearing bore for an adjusting wrench engaging the circumferential toothing. It is preferably provided that the adjustment nut liftably rests with its upper edge on a front face of the outer rod and cooperates therewith without undercut.

The gist of this aspect of the invention is the idea that only a very small, patient-friendly access opening is required when using the bearing bore for the adjusting wrench. It is possible therewith to frequently readjust the length and—especially in younger patients—adapt it to growth. It is easily possible, due to the modular construction, to exchange the length-adjusting mechanism for a larger one when there is no more room for adjustment.

Due its being preferably mounted in a free-floating manner, the adjustment nut is axially displaceable relative to the outer rod, namely it only rests on the front side thereof without being secured there by a positive fit guidance, particularly an undercut; the adjustment nut can thus be freely moved away from the outer rod.

Two substantial advantages are associated with this construction. On the one hand, it allows the parts of the prosthesis to be separated from each other. The wound required for implanting the tubular bone prosthesis may therefore be considerably smaller. This is clearly less onerous for the patient and easier to handle for the surgeon.

Another advantage is that, due to the feature of the adjustment nut being mounted without undercut, a greater force application surface becomes possible between the adjustment nut and the outer rod on the front face. Due to this greater force application surface, the prosthesis is thus subject to less strain and/or can be designed to be smaller and thus slimmer whilst offering the same robustness. It is precisely this last feature that constitutes a significant advantage as regards implantation in young patients.

It is known to adjust the length of the tubular bone substitute in a prosthesis for replacing a tubular bone including an adjacent joint by providing a bevel gear at the transition between joint and tubular bone (U.S. Pat. No. 4,892,546). This admittedly offers the advantage of enabling adjustment of length without requiring a major surgical intervention. It is, however, a disadvantage that the required bevel gear is comparatively bulky. Therefore, this prosthesis is less suitable for application in young patients, particularly in children. Furthermore, a tubular bone prosthesis is known which has a telescopic shaft including a shaft and a sleeve, with a union nut being provided on the sleeve (U.S. Pat. No. 4,502,160). The union nut is axially guided on the sleeve, fixed thereto in a positive fit manner, so as to be able only to rotate, but not to move in the longitudinal direction. With its internal thread, the union nut cooperates with an external thread disposed on the shaft. The length can be changed by rotating the union nut. Due to the fact that the union nut is fixed to the sleeve in a positive fit manner, the prosthesis can only be implanted when fully assembled. This complicates implantation since a large access opening is required for the fully assembled prosthesis. As a result, the surgical wound thus becomes disproportionately large, which may represent a heavy burden especially to the group of young patients.

The prosthesis according to the invention is hence significantly less onerous for patients and is more advantageous as regards growth behaviour, it being thus particularly suitable for treating young patients (children) during their growth period. The reason is that the growth plate of the bone must frequently be resected during the implantation process. The prosthesis according to the invention is, however, perfectly suitable also for application in adults experiencing postsurgical changes, for example due to ligament lengthening.

The circumferential toothening is preferably designed as a steep toothing. Steep toothing is understood to mean that the load-bearing flanks include a flank angle of from at least 50° to no more than 85°, preferably at least 60°. Such a steep orientation of the load-bearing flanks suppresses or largely avoids the generation of axial force due to actuation of the adjusting wrench and action thereof on the circumferential toothing of the adjustment nut. Undesired parasitic adjustment of length or undesired axial displacement caused by the adjusting wrench can thus be avoided. It is hence ensured that the adjustment of length is based solely on the axial displacement resulting from rotational movement of the adjustment nut due to the lead of the internal thread of the adjustment nut.

Preferably the toothing is embedded in an appropriate circumferential recess. Here, the recess is preferably formed on the outer edge of the upper side. By this means, the circumferential toothing does not protrude, i.e. no crests stick out in the axial direction. This efficiently obviates the risk of causing irritation to the surrounding tissue.

The following moreover applies to all of the embodiments:

The internal thread of the adjustment nut preferably is a single-start thread. Here, “single-start” is understood to mean that there is just one thread which is continuous from one side of the nut to the opposite side. As a result of there being only one thread, it is possible to position the adjustment nut in a defined manner relative to the inner rod in the direction of rotation. This simplifies accurate alignment and thus length adjustment, ruling out the risk of positional ambiguities.

Preferably the thread of the inner rod is flattened. Here, “flattened” is understood to mean that the crests of the thread on the inner rod are chamfered, i.e. not pointed in the narrower sense of the term, but are rather replaced by a preferably flat area. This flat area as a whole forms a hollow cylindrical shell. The thread of the inner rod therefore has less sharp edges affecting its surroundings. This reduces the risk of irritation.

The adjustment nut advantageously has a polished peripheral surface. This prevents the surrounding tissue from adversely affecting the adjustment nut, and so there is hardly any adhesion. The adjustment nut thus remains adjustable even many years after implantation and is not blocked by tissue (connective tissue) growing over it. The polished peripheral surface can also be achieved by designing this surface in any other manner leading to reduced adhesion. Anodizing of the surface can be considered here, particularly in case of titanium endoprostheses.

The adjustment nut expediently has a plurality of radial holes on its peripheral surface which are preferably arranged at a regular angular distance. These radial holes are for accommodating an adjusting pin. This is inserted into one of these holes, thus allowing the adjustment nut to be rotated by a specific angular amount until the adjusting pin has reached its stop position. By reinserting the adjusting pin into one of the other radial holes which are preferably arranged at equal angles, it can be actuated again, with the result of achieving a turning of the adjustment nut and, thus, an adjustment of length. This also offers the advantage of permitting emergency actuation if the length-adjusting mechanism cannot be actuated by means of the adjusting wrench.

The adjustment nut expediently has a rounded tactile marking. This makes it possible to exactly define a “zero position” of the adjustment nut in the direction of rotation. This is expedient where the length to be readjusted or the growth in length is transferred mathematically, measured in rotations of the adjustment nut. In order to have a zero position here, the tactile marking is of great advantage. Expediently the outer rod, which has the adjustment nut abutting its front side, comprises an identically shaped continuation of the tactile marking. A harmonic transition thus results between the tactile marking on the adjustment nut and the continuation on the outer rod. This efficiently obviates the risk of causing irritation to surrounding tissue.

The inner rod advantageously has recesses provided thereon in which a latching member is engaged that is disposed on the outer rod. These recesses may be a series of bores disposed on the outside of the shaft. They are expediently disposed in an axial groove. Their purpose is to receive a screw being screwed therein which with its tip engages in the recess, thereby securing the inner rod against accidental axial movement. Inadvertent separation of the inner rod from the outer rod is safely avoided by this means and can thus be counteracted. The fastening screw is advantageously designed as a grub screw. While requiring little space, it can still ensure a sufficiently safe locking of the length-adjusting mechanism.

Preferably at least one of the two elements, i.e. the thread of the inner rod and/or the internal thread of the adjustment nut, consists of titanium-free material, particularly cobalt-chromium material. This offers the advantage—specifically in combination with titanium, the material of choice in the fabrication of prostheses—that thread seizure will not occur. Protection against inadvertent blocking of the thread, particularly due to seizure, represents a significant advantage for the tubular bone prosthesis according to the invention, the most important property of which is its longitudinal displaceability. The invention will now be explained with reference to the enclosed figures showing advantageous example embodiments, in which:

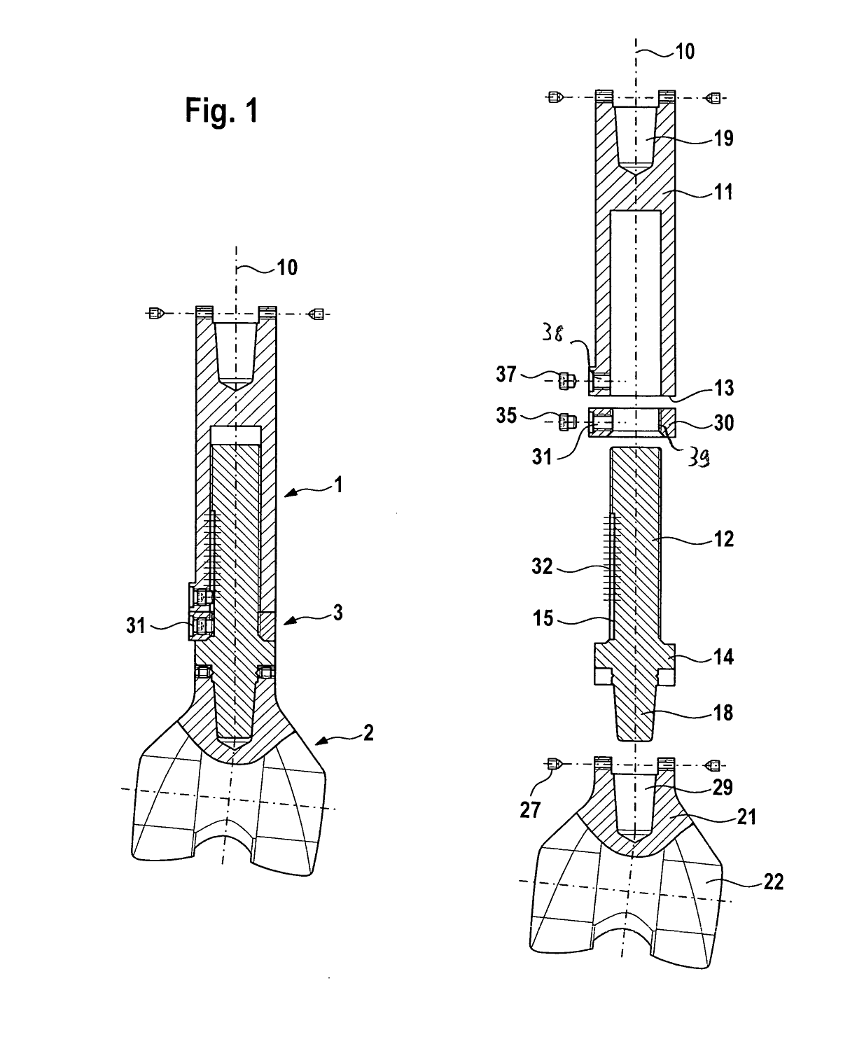

FIG. 1 is a sectional view of a knee joint prosthesis according to a first example embodiment of the invention;

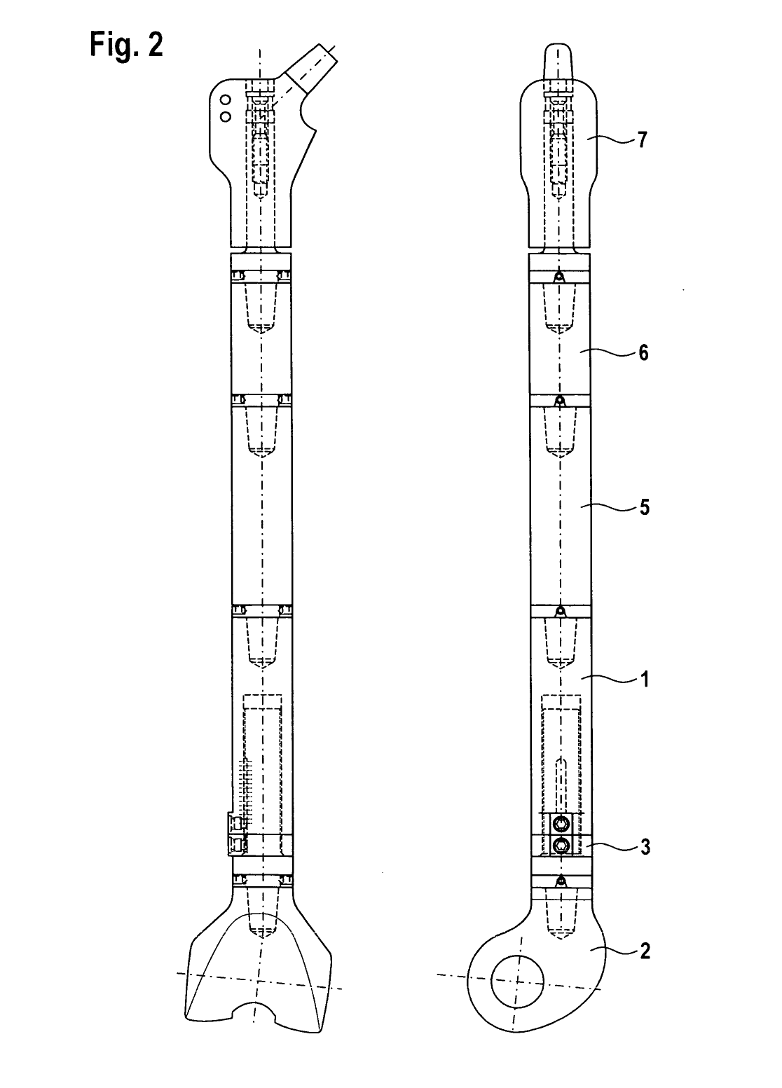

FIG. 2 is a front and lateral view of a total prosthesis based on the first example embodiment according to FIG. 1;

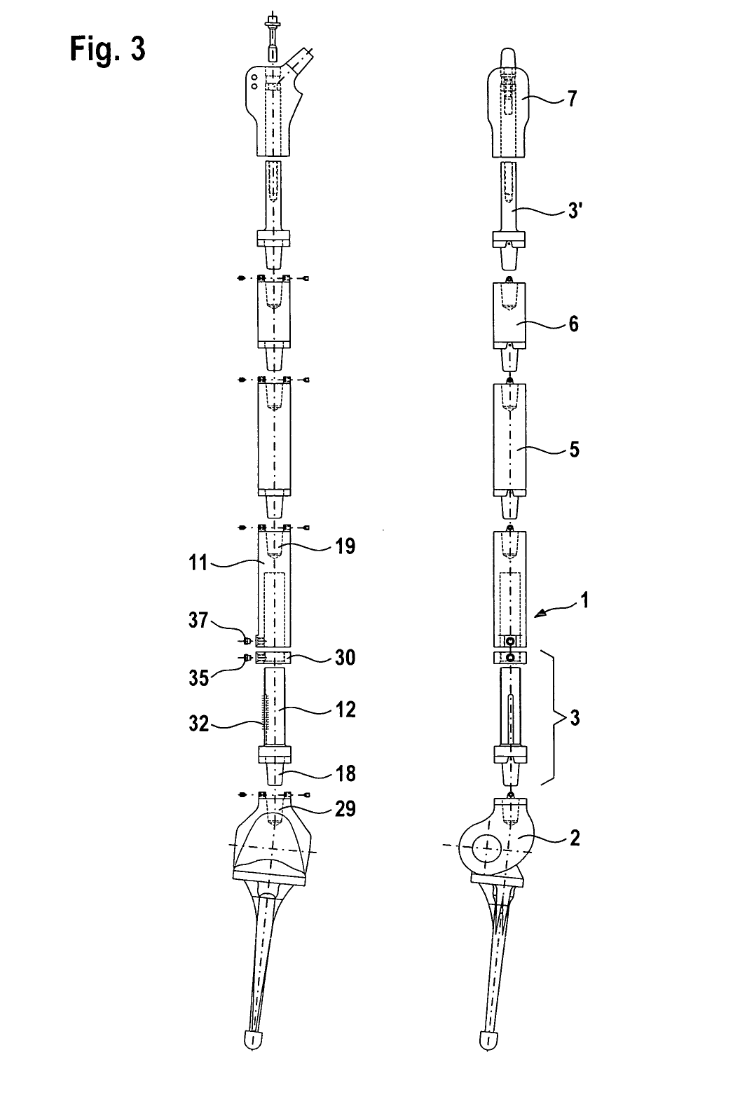

FIG. 3 shows exploded views of FIG. 2;

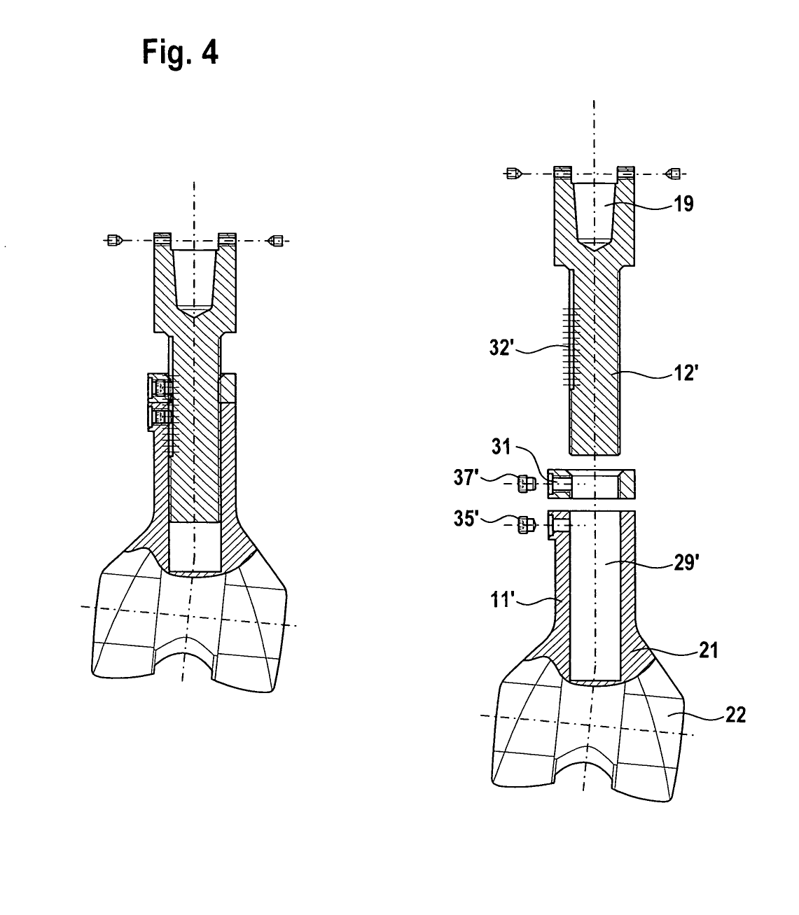

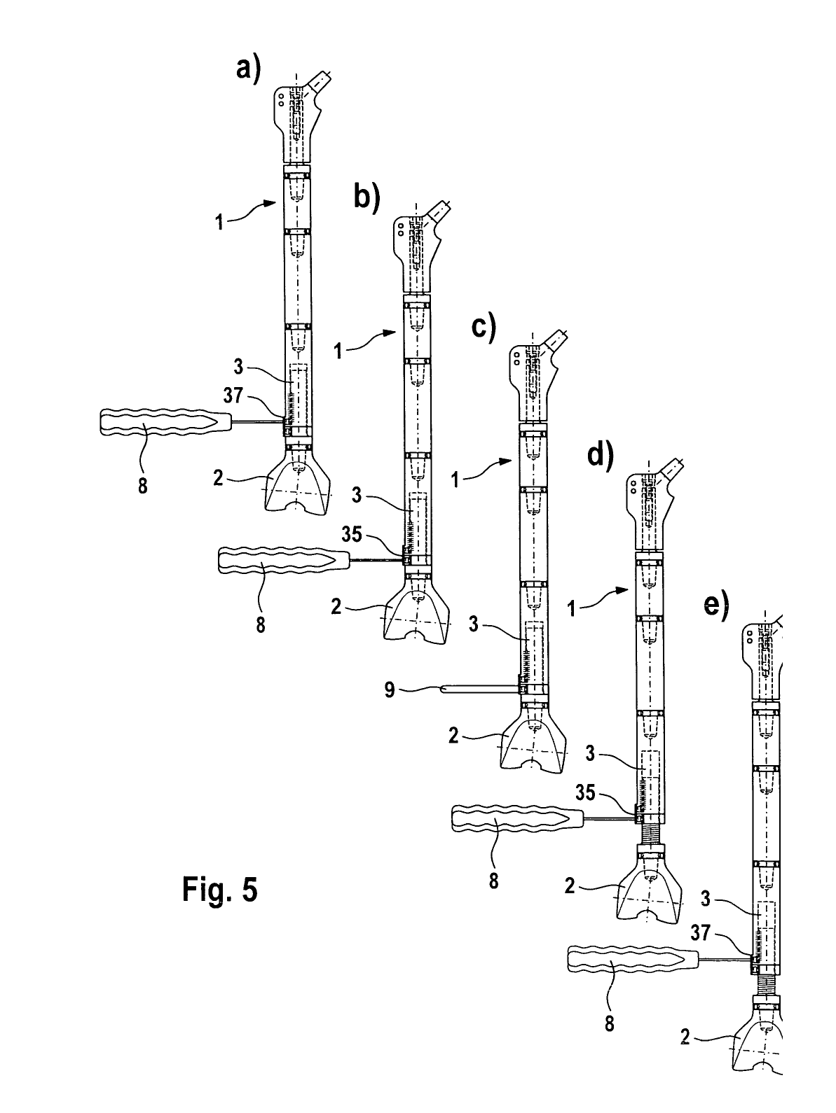

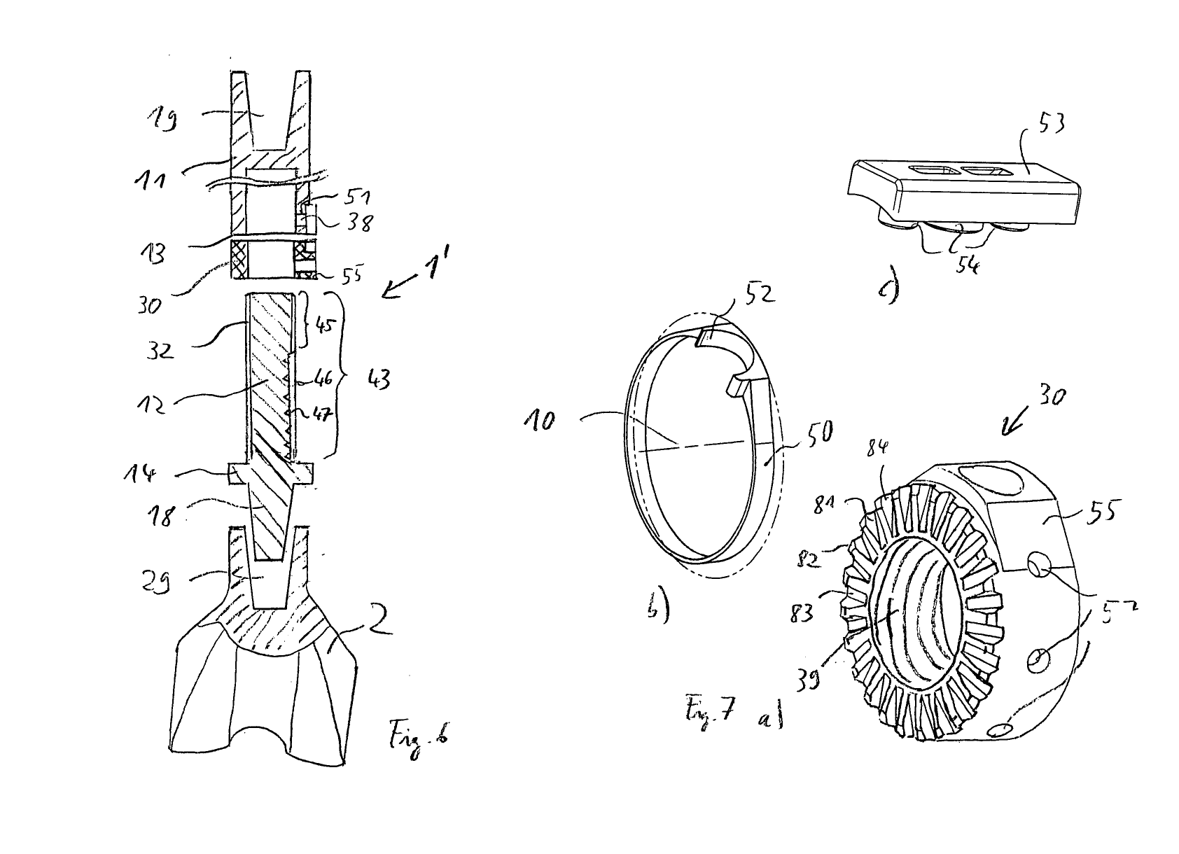

FIG. 4 is a sectional view of a variant;

FIGS. 5a-e show how length adjustment is performed;

FIG. 6 is an exploded view of a second example embodiment of the invention;

FIGS. 7a-c are enlarged detailed views of the second example embodiment;

FIG. 8 shows the function of a length-adjusting mechanism;

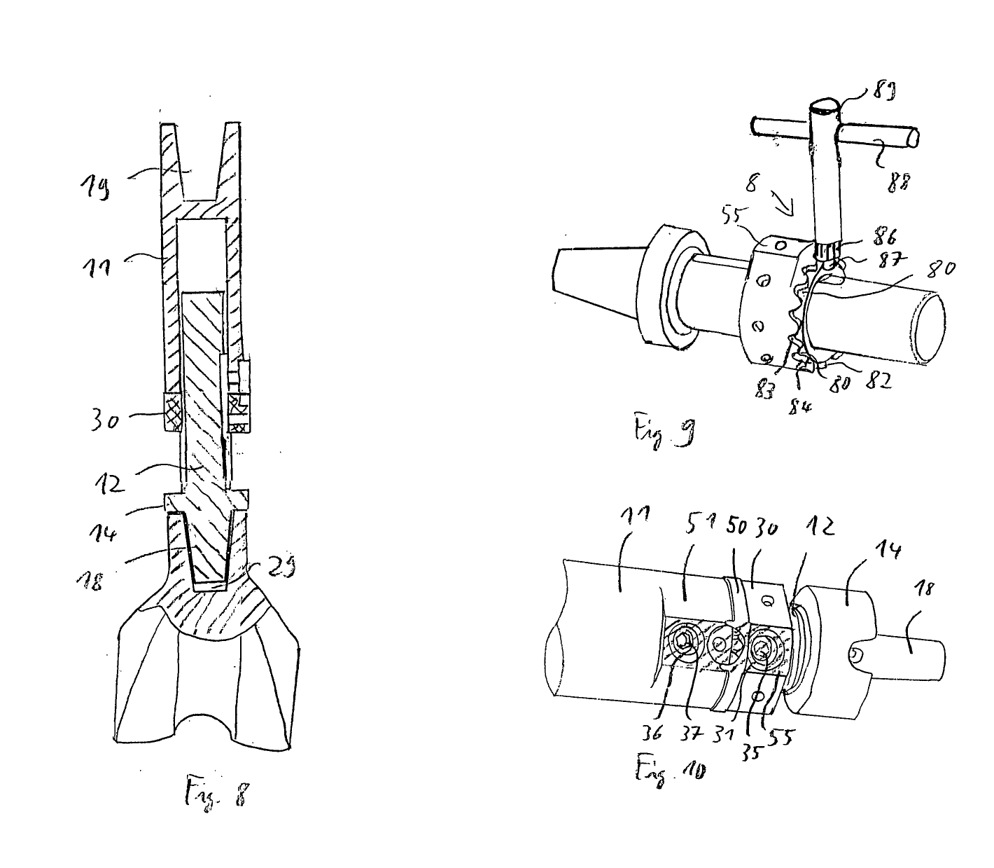

FIG. 9 is a fully assembled view; and

FIG. 10 is a perspective view.

FIG. 1 shows an example embodiment of the prosthesis according to the invention which is intended as a joint prosthesis for replacing part of the knee and part of the distal femur. It comprises, as components, a shaft 1, a joint mechanism 2 and a length-adjusting mechanism 3. The shaft 1 comprises an outer rod 11 and an inner rod 2 guided inside the outer rod 11 so as to be telescopically displaceable along its central axis 10. The outer rod 12 has at its first end a female cone connector 19 which is for coupling further rod segments (not shown in FIG. 1) as needed; it should be noted that it may also be sealed by a blind plug or may be dispensed with altogether. At its second end the outer rod has a front flange 13 with a radially oriented front face. The inner rod 12 has at its first end a complementary, male cone connector 18 adapted to engage in a matching female cone connector 29 on the joint mechanism 2. At the transition to the cone connector 18, the inner rod 12 has a collar 14, one end face of which, facing the fixed end, serves as a stop for the cone connector 18, and the other face of which, facing the shaft of the inner rod 12, serves as a stop for an adjustment nut 30.

The actuating mechanism 3 comprises the adjustment nut 30 having a single-start internal thread 39 which meshes with a single-start adjustment thread 32 disposed on the inner rod 12. The adjustment nut 30 has at its lateral faces a plurality of engagement apertures 31 configured as radial bores. They are adapted to receive a pin 9 (see FIG. 5c) as an actuating member. This is used to rotate the adjustment nut 30 relative to the inner rod 12 having the mating thread 32, by means of which the adjustment nut 30 moves along the central axis 10. In its initial position, the adjustment nut 30 is directly contiguous with the front flange 13 of the outer rod 11 and takes the latter with it as it moves. This causes the outer rod 11 to move along the longitudinal axis 10 relative to the inner rod 12 such that the distance between the adjustment nut 30 and the collar 14 increases, as does the overall length of the shaft 1. When the adjustment nut 30 is rotated in the opposite direction, the process takes place in the reverse direction and the overall length becomes shorter.

Securing mechanisms are provided to fix a set length. These include an adjustment lock 35 and an anti-rotation means 37. The adjustment lock 35 comprises a clamping screw which is inserted in one of the radial bores 31 of the adjustment nut 30 and acts with its tip on a flat portion 15 on the outer rod 12. As a result, the adjustment nut 30 is locked in a positive fit manner. It is thus ensured with certainty that even under heavy and frequently varying loads there can be no inadvertent rotation of the adjustment nut 30 with a corresponding change of length. The anti-rotation means 37 is similar in structure and has a fixing screw disposed in a radial bore in the region of the front flange 13 on the outer rod. This fixing screw acts with its tip in the region of the mating thread 32, thus securing the outer rod 11 against rotation relative to the inner rod 12. Accidental rotation of the inner rod 12 relative to the joint mechanism 2 is in turn prevented by means of two diametrically opposed fixing screws of a conical lock 27 that is known per se. As a result, rotation is prevented continuously from the joint mechanism 2 to the length-adjusting mechanism 3 and the shaft 1.

The length adjustment process is illustrated in FIG. 5 on the example of an implanted prosthesis that is to be adjusted to greater length so as to accommodate for the patient's growth. This requires first of all that the prosthesis be accessed by means of minimally invasive surgery. A stab incision is normally sufficient for this. In a first step (FIG. 5a), a screwdriver 8 is slid through the incision and is engaged with the securing screw for the anti-rotation means 37. The anti-rotation lock is released by unscrewing the screw. In a second step (FIG. 5b), the adjustment lock 35 is released in just the same way. The length-adjusting mechanism 3 thus becomes disengaged and can be actuated. The screwdriver 8 is removed and an adjusting pin 9 is inserted through the incision and engaged with one of the radial bores 31 of the adjustment nut. By swivelling the pin 9, the adjustment nut 30 is rotated to some extent, then the pin 9 is reinserted into an adjacent radial bore and the adjustment nut 30 is rotated some more. In the example embodiment shown, the thread lead is selected such that a change in length of 2 mm results per revolution of the adjustment nut 30. Once the desired length has been set, the pin 9 is withdrawn and the screwdriver 8 is introduced once again to successively reinstall, and thus reactivate, the adjustment lock 35 (FIG. 5d) and the anti-rotation means (FIG. 5e).

The embodiment illustrated in FIG. 1 shows a basic prosthesis. This can be supplemented with additional elements, as depicted in FIGS. 2 and 3. There, additional shaft segments 5, 6 are provided which are joined via cone connectors matching the cone connectors 18, 19 of the shaft 1 and 29 of the joint mechanism 2 so as to form a long shaft (see exploded view in FIG. 3). At the upper end thereof, there is arranged a femoral neck prosthesis 7. A total femoral prosthesis is thus formed which, unlike in the prior art, cannot just be designed to have graduated lengths but is rather, thanks to the modular length-adjusting mechanism 3, even steplessly adjustable. This allows for fine adjustment. With the length-adjusting mechanism 3 in an initial position (as shown in FIG. 1), the shaft 1 preferably is the same length as one of the shaft segments, for example the shaft segment 5. As a result, a prosthesis system is provided in which an adjustable-length or fixed-length shaft can be formed, as needed, by simply replacing the elements 1, 5.

An alternative embodiment is illustrated in FIG. 4, wherein identical elements are identified by the same reference numbers. The difference to the first example embodiment essentially lies in that the outer rod 11′ and the inner rod 12′ are inversely arranged, i.e. the outer rod 11′ is arranged on the joint mechanism 2 and the inner rod 12′ forms the first end with the cone connector 19. Such an inversely designed length-adjusting mechanism 3′ may also be provided on the first end of a long shaft having several shaft segments 5, 6, as shown in FIG. 3.

Reference will now be made to the second example embodiment as shown in FIGS. 6 to 10. This comprises a particular actuating mechanism for the length-adjusting mechanism. Elements of the same type are identified by the same reference numbers. An insertion area 43 extends from the collar 14 to the other, free end of the shaft 12. This insertion area comprises the external thread 32. This is a single-start thread and the cross-sectional shape of the individual threads is substantially triangular with a flattened crest. Furthermore, the inner rod comprises, except in a short guidance portion 45 which approximately corresponds to 1.5 times the rod diameter, a longitudinal groove 46 having configured at its bottom a number of blind holes 47 in a line oriented in parallel to the longitudinal axis.

A tactile marking 55, configured as an elevation, is disposed on the substantially smooth outer surface of the adjustment nut 30 (see FIG. 7a). Eight counterbores 57 are also disposed on the outer surface at a regular angular distance and in a uniform radial plane, one of which being disposed in the tactile marking 55. At its lower edge facing the inner rod 12, the adjustment nut 30 is designed to be complementary to the collar 14 and includes a planar outer contact surface 52. The internal thread 39 is disposed in a portion of the adjustment nut 30 that is made from cobalt chrome (CoCr); preferably the entire adjustment nut 30 consists of cobalt-chromium material.

The adjustment nut has at its upper edge a circumferential toothing 81 which is part of an actuating mechanism 8. The toothing 81 has an undulating profile with rounded crests 82 and roots 83. The flanks 84 joining the crests 82 and the roots 83 are designed as steep flanks having in their central part an inclination (based on the radial plane as defined by the upper edge 56) of 60°. The roots 83 ascend from the outside to the inside, resulting in a conically tapered tooth structure, as is suitable particularly for a right-angle gear drive. The toothing 81 is disposed on a recess 80 extending along the outside of the upper edge 56, such that the crests 82 do not protrude, but finish flush with the plane defined by the upper edge (see FIG. 9; outer rod not shown for the sake of greater clarity). As a result, there is some kind of double-shell structure on the upper edge, with a circumferential inner ring forming a planar and undercut-free contact surface as an inner shell, and with the toothing 81, the rounded crests 82 of which finish flush and level with the inner ring 56′, as the outer shell.

The front flange 13 of the outer rod 11 is substantially planar, it being in particular devoid of undercuts, i.e. nowhere is there an undercut. On the outer surface of the outer rod 11, there is disposed a second protrusion 51 adjacent to the edge. It has a radial bore 38 acting as a bearing seat for an adjusting wrench 89 of the actuating mechanism 8. The distance between the radial bore 38 and the front flange 13 is adapted to the dimensions of the adjusting wrench 89, as will be described in more detail in the following.

A grub screw 37 can be provided as a locking instrument on the outer rod 11. It is preferably capable of being screwed into the bearing bore 38 and protrudes with its tip, in the screwed-in position, into the longitudinal groove 46, to be precise into one of the blind holes 47, thus securing the inner rod 12 against undesired dislocating movements.

The adjusting wrench 89 is structured in the same manner as a bevel gear key as is known for actuating chucks. It comprises at its rear end an actuating handle 88 which, in the simplest case, may be a cross bar. At the front end there is provided a conical toothing 86 which is designed so as to be able to mesh with the toothing 81 of the adjustment nut 30. To engage the conical toothing 86 with the toothing 81, a bearing pin 87 is formed on the front tip that is designed to be complementary to the radial bore 38, with the result that a pivot bearing is formed. The distance from the radial bore 38 to the front flange 13 is adapted to the diameter of the conical toothing 96 in such a manner that, when the adjusting wrench 89 is inserted in the radial bore 38, the conical toothing 86 is engaged with the toothing 81 of the adjustment nut 30 which with its upper edge lies flush against the front flange 13 of the outer rod 11.

The actuating mechanism 8 is actuated as follows. In its initial state, the adjustment nut 30 is screwed onto the external thread 32 of the inner rod 12. The latter is pushed into the outer rod 11 until the upper edge of the adjustment nut 30 lies flush against the front flange 13 of the outer rod 11. By turning the adjusting wrench 89, which is inserted in the bearing bore 31, the conical toothing 86 thereof meshes with the toothing 81 of the adjustment nut 30, by means of which the latter is turned and the inner rod 12 is pushed out of the outer rod 11. The pushing distance is determined here by the lead of the external thread 32 cooperating with the adjustment nut 30 and by the gear ratio between the conical toothing 86 and the toothing 81. During adjustment, the adjustment nut 30 remains contiguous with the outer rod 11.

If growth of the patient (or a lengthening in supporting ligaments) gives rise to femoral lengthening, the endoprosthesis according to the invention can be adapted thereto. This is done by readjusting the adjustment nut 30. It is sufficient for this to just insert the driving tool 89 into the bearing bore 38 by means of a minor and thus patient-friendly intervention, and the adjustment nut 30 is readjusted by turning. The amount of readjustment is unambiguously determined by the number of revolutions of the adjusting wrench 89. In order to be easily able to control the number of revolutions, there is provided the tactile marking 55 on the adjustment nut 30. In its initial position, it is flush with the protrusion 51 of the same kind on the outer rod 11 and it always returns to the flush position whenever the adjustment nut 30 has made one complete revolution. This makes it possible to easily verify, by touch, the correct position from the outside as well.

In order to ensure proper functioning of the actuating mechanism 8 even after long-term implantation, there are provided a toothing protector ring 50 having a moulded-on section 52 for the conical toothing 86 of the adjusting wrench 89 and a multi cover 53 including several (i.e. three in the example embodiment shown) pin stubs 54 (see FIGS. 7b, c). The toothing protector ring is disposed between the adjustment nut 30 and the front flange 13 of the outer rod 11 and covers the toothing 81 at the outside. This prevents tissue growing into the toothing and the associated risk of blockage. To further prevent growth into the radial bores 31, 38, there is provided the multi cover 53. This is a substantially cuboid block inserted with its pin stubs 34 into the radial bores 31, 38 and held clamped therein. It covers the area shown hatched in FIG. 10 and thus reliably prevents undesired growth of tissue. For length adjustment, it only needs to be removed so as to allow unrestricted access to the screws 35, 37 as well to the toothing 81.

To receive the bearing pin 87 of the adjusting wrench 89, there is provided, in the variant shown in FIG. 10, a discrete bore that is distinct from the bore in which the screw 37 is received to offer protection against rotation (instead of the combined design as shown in FIGS. 1 to 8).

1. A prosthesis for replacing at least part of a tubular bone and an adjoining joint, comprising an elongate shaft with a first and a second end, and a joint mechanism arranged at the second end of the shaft,
wherein a length-adjusting mechanism is provided which actuates the shaft along its axis in the manner of a telescope, wherein the shaft and the joint mechanism are coupled via complementary connectors, wherein the length-adjusting mechanism is of a modular design and is provided, at its proximal and distal ends, with the complementary connectors and is further provided with an anti-rotation means acting in a positive fit manner.

2. The prosthesis according to claim 1, wherein the shaft comprises an inner rod and a coaxial outer rod which are acted upon by the length-adjusting mechanism and in that the anti-rotation means is arranged on the outer rod and engages in a longitudinal recess on the inner rod.

3. The prosthesis according to claim 2 wherein a dual securing mechanism is provided which comprises an adjustment lock in addition to the anti-rotation means.

4. The prosthesis according to claim 3, wherein the anti-rotation means is non-rotatably disposed and the adjustment lock is rotatably disposed.

5. The prosthesis according to claim 4, wherein a compression flange is provided which has two opposite collar faces, one of which is a thrust bearing for the connectors and the other forms a stop for length adjustment.

6. The prosthesis according to claim 5, wherein a second length-adjusting mechanism is provided for the shaft.

7. The prosthesis according to claim 6, wherein the length-adjusting mechanism comprises a screw drive which is encapsulated in the initial position.

8. The prosthesis according to claim 7, wherein a mechanism for actuating the length-adjusting mechanism is provided having a thread on the inner rod and an adjustment nut screwed onto the thread and having a circumferential toothing, wherein a bearing bore is provided on the outer rod for an adjusting wrench engaging in the circumferential toothing.

9. The prosthesis according to claim 8, wherein the adjustment nut liftably rests with its upper edge on a front flange of the outer rod and cooperates therewith without undercut.

10. The prosthesis according to claim 9 wherein the circumferential toothing is designed as a steep toothing, the load-bearing flanks of which have a flank angle of at least 50 degrees to no more than 85 degrees, preferably at least 60 degrees.

11. The prosthesis according to claim 10 wherein the toothing is arranged in a circumferential recess.

12. The prosthesis according to claim 11, wherein a protective ring, preferably made from resilient synthetic material, is provided which is arranged between the adjustment nut and the front flange and covers the toothing at the outside.

13. The prosthesis according to claim 12, wherein the internal thread of the adjustment nut is a single-start thread.

14. The prosthesis according to claim 13, wherein the thread of the inner rod is flattened.

15. The prosthesis according to claim 14, wherein the adjustment nut has a polished perimetral face.

16. The prosthesis according to claim 15, wherein the adjustment nut has on its perimetral face a plurality of radial holes, preferably at a uniform angular distance.

17. The prosthesis according to claim 16, wherein the adjustment nut has a rounded tactile marking which is continued, preferably identical in shape, on the outer rod.

18. The prosthesis according to claim 17, wherein a plurality of recesses are provided on the inner rod into which a locking member arranged on the outer rod engages.

19. The prosthesis according to claim 18, wherein the thread and/or the internal thread consist of titanium-free material, particularly cobalt-chromium material.

20. A prosthesis system comprising a prosthesis including an elongate shaft with a first and a second end, and a joint mechanism arranged at the second end of the shaft, wherein a length-adjusting mechanism is provided which actuates the shaft along its axis in the manner of a telescope, wherein the shaft and the joint mechanism are coupled via complementary connectors, wherein the length-adjusting mechanism is of a modular design and is provided, at its proximal and distal ends, with the complementary connectors and is further provided with an anti-rotation means acting in a positive fit manner; and several shaft members of different lengths coupled via the connectors.

21. The prosthesis system according to claim 20, wherein one of the shaft members is the same length as the length-adjusting mechanism in its initial position.
